SEQUENCE LISTING (28 sequences)

SEQ ID NO: 1 - RNA, 22 nt, Homo sapiens
acugcagugagggcacuuguag

SEQ ID NO: 2 - RNA, 23 nt, Homo sapiens
caaagugcuuacagugcagguag

SEQ ID NO: 3 - RNA, 23 nt, Homo sapiens
uaaggugcaucuagugcagauag

SEQ ID NO: 4 - RNA, 22 nt, Homo sapiens
aguuuugcauaguugcacuaca

SEQ ID NO: 5 - RNA, 22 nt, Homo sapiens
aguuuugcagguuugcauuuca

SEQ ID NO: 6 - RNA, 23 nt, Homo sapiens
uaaagugcuuauagugcagguag

SEQ ID NO: 7 - RNA, 22 nt, Homo sapiens
ggguggggauuuguugcauuac

SEQ ID NO: 8 - RNA, 23 nt, Mus musculus
caaagugcuuacagugcagguag

SEQ ID NO: 9 - RNA, 22 nt, Mus musculus
caaagugcuuacagugcaggua

SEQ ID NO: 10 - RNA, 23 nt, Mus musculus
uaaggugcaucuagugcagauag

SEQ ID NO: 11 - RNA, 22 nt, Mus musculus
uaguuuugcauaguugcacuac

SEQ ID NO: 12 - RNA, 23 nt, Mus musculus
aguuuugcagguuugcauccagc

SEQ ID NO: 13 - RNA, 23 nt, Mus musculus
uaaagugcuuauagugcagguag

SEQ ID NO: 14 - RNA, 23 nt, Mus musculus
agguugggauuugucgcaaugcu

SEQ ID NO: 15 - DNA, 23 bp, Artificial Sequence
  Primer
gcggtctggcagtaaaaactatc

SEQ ID NO: 16 - DNA, 23 bp, Artificial Sequence
  Primer
gtgaaacagcattgctgtcactt

SEQ ID NO: 17 - DNA, 20 bp, Artificial Sequence
  Primer
tcgagtatctgacaatgtgg

SEQ ID NO: 18 - DNA, 21 bp, Artificial Sequence
  Primer
tagccagaagttccaaattgg

SEQ ID NO: 19 - DNA, 21 bp, Artificial Sequence
  Primer
atagcctgaaaccaactgtgc

SEQ ID NO: 20 - DNA, 20 bp, Artificial Sequence
  Primer
atcgtgtacattggcttgcg

SEQ ID NO: 21 - DNA, 18 bp, Artificial Sequence
  Primer
actgtggcgtggcmttmc

SEQ ID NO: 22 - DNA, 21 bp, Artificial Sequence
  Primer
acamgccagagtccttcagag

SEQ ID NO: 23 - DNA, 19 bp, Artificial Sequence
  Primer
gcaggtggtacgttgtggg

SEQ ID NO: 24 - DNA, 22 bp, Artificial Sequence
  Primer
gacaggattgacagattgatag

SEQ ID NO: 25 - DNA, 20 bp, Artificial Sequence
  Primer
atttctgcatgctgctggtg

SEQ ID NO: 26 - DNA, 20 bp, Artificial Sequence
  Primer
acatggcagtttctgagcag

SEQ ID NO: 27 - DNA, 21 bp, Artificial Sequence
  Primer
gcccagtgttaccaccmgaag

SEQ ID NO: 28 - DNA, 23 bp, Homo sapiens
ugugcaaauccaugcaaaacuga

(19) United States
(12) Patent Application Publication
(10) Pub. No.: US 2023/0323347 A1
(43) Pub. Date: Oct. 12, 2023

(54) miR-17˜92 for Treatment or Protection Against Acute Kidney Injury

(71) Applicant: University of Pittsburgh - Of the Commonwealth System of Higher Education, Pittsburgh, PA (US)

(72) Inventors: Sunder Sims-Lucas, Allison Park, PA (US); Takuto Chiba, Pittsburgh, PA (US); Jacqueline Ho, Pittsburgh, PA (US)

(21) Appl. No.: 18/025,299

(22) PCT Filed: Sep. 17, 2021
(86) PCT No.: PCT/US2021/050885

§ 371 Date: Mar. 8, 2023

Related U.S. Application Data
(60) Provisional application No. 63/080,120, filed on Sep. 18, 2020.

(51) Int. Cl. C12N 15/113; A61P 13/12

(52) U.S. Cl. CPC C12N 15/113; A61P 13/12; C12N 2310/141

(57) ABSTRACT

This application discloses a method for treating a human patient. The method includes administering to the patient a pharmaceutical composition having one or more miRNA of the miR-17˜92 cluster, or an miRNA mimic of an miRNA of the miR-17˜92 cluster. The patient has at least one medical condition that places him or her at an increased risk of developing AKI, incipient AKI, or a sequelae of AKI.

### CROSS REFERENCE TO RELATED APPLICATIONS

This application claims priority to U.S. Provisional Application US 63/080,120, filed Sep. 18, 2020, the disclosure of which is incorporated herein by reference in its entirety.

### STATEMENT REGARDING FEDERAL FUNDING

This invention was made with government support under Grant No. DK125015 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Sep. 13, 2021, and modified on Sep. 16, 2021, is named “Sequence Listing.txt”, and is 4,865 bytes in size.

The present disclosure relates to the field of pharmaceutical compositions. More specifically it pertains to the use of microRNA (miRNA) clusters as potential therapeutic agents for the treatment of acute kidney injury (AKI).

MicroRNAs (miRNAs) are endogenous single-stranded noncoding mRNAs of 20-25 nucleotides that play critical roles in the post-transcriptional regulation of multiple biological cell functions, including proliferation, differentiation, metabolism and apoptosis. In AKI, some miRNAs appear to act pathogenically by promoting inflammation, apoptosis, and fibrosis, while others may act protectively by exerting anti-inflammatory, anti-apoptotic, anti-fibrotic, and pro-angiogenic effects. Furthermore, miRNAs have emerged as promising therapeutic targets in various diseases, including kidney fibrosis and diabetic kidney disease with many ongoing clinical trials. However, the therapeutic implication of miRNAs in AKI has not yet been explored.

The miR-17˜92 cluster is a polycistronic miRNA that comprises seven individual mature miRNAs: miR-17-3p, miR-17-5p, miR-18a, miR-19a, miR-19b, miR-20a, and miR-92a (FIG. 16). This cluster is a known oncogene and has been implicated in multiple different medical conditions, including, but not limited to, neurological diseases, heart disease, cancer, and abnormal bone development. The cluster is also highly expressed in embryonic cells and decreases after differentiation.

AKI most commonly occurs in a hospital setting. Hospital-acquired AKI accounts for 22% of all cases worldwide, and nearly 50% of critically ill inpatients are estimated to suffer from AKI. AKI is associated with high rates of morbidity and mortality, and causes two million deaths per year. While the kidney may at least partially recover, the patients are at an increased risk for subsequently developing chronic kidney disease (CKD); other times, the acute injury is so severe that there is no kidney recovery leading to end stage renal disease (ESRD).

Although research advances have been made in recent decades, a definitive and effective treatment for AKI is still lacking, nor are there available interventions to decrease the risk of progression to CKD after AKI. The best strategies currently focus on prevention, early diagnosis and early interventions aimed at managing the underlying etiologies and complications of AKI. The widely accepted diagnostic criteria for AKI are based on changes of serum creatinine (sCr) and urine output; however, obvious changes in SCr may not be seen until 48-72 hrs after renal insult, potentially delaying the diagnosis of AKI. To enable the early diagnosis of AKI, researchers have investigated various novel biomarkers, such as neutrophil gelatinase-associated lipocalin (NGAL) and kidney injury molecule-1 (KIM-1). However, these biomarkers may be affected by diseases other than AKI, and there are controversies regarding cut-offs. To date, researchers have been unable to establish a biomarker-guided clinical strategy that reliably improves the clinical outcome in patients with AKI. One of the hallmarks of AKI is damage to the renal microvasculature. This damage alters endothelial function, contributing to hypoxic and inflammatory injury to the renal parenchyma. Although an angiogenic response (vascular sprouting from existing vessels) is often relevant to endothelial cell repair (and therefore AKI recovery), the renal microvasculature has been considered to have a limited reparative capacity.

Thus, there is a need for improved methods to treat or reduce the severity of AKI after is has occurred and to prevent AKI even before it happens.

### SUMMARY

Disclosed herein is a method of treating a human patient. The method comprises administering to the patient a pharmaceutical composition comprising at least one miRNA of the miRNA-17˜92 cluster, or an miRNA mimic of an miRNA of the miRNA-17˜92 cluster; wherein said patient has at least one medical condition chosen from acute kidney injury (AKI), a medical condition associated with an increased risk of developing AKI, incipient AKI, or a sequelae of AM.

Also disclosed herein is a pharmaceutical composition comprising at least one miRNA, or mimic thereof, and at least one liquid, non-aqueous, pharmaceutically acceptable excipient.

Also disclosed herein is a pharmaceutical composition for use in treating AKI. The composition comprises a miR-18a mimic, a miR-19b mimic, and at least one liquid, non-aqueous, pharmaceutically acceptable excipient.

The following numbered clauses describe various aspects or embodiments of the present invention.
Clause 1. A method of treating a human patient, the method comprising:
administering to the patient a pharmaceutical composition comprising at least one miRNA of the miRNA-17˜92 cluster, or an miRNA mimic of an miRNA of the miRNA-17˜92 cluster;
   wherein said patient has at least one medical condition chosen from acute kidney injury (AKI), a medical condition associated with an increased risk of developing AKI, incipient AM, or a sequelae of AM.
Clause 2. The method of clause 1, wherein said patient has renal ischemia.
Clause 3. The method of clause 1, wherein said patient has a myocardial infarction or congestive heart failure.
Clause 4. The method of clause 1, wherein said patient has sepsis or shock.
Clause 5. The method of clause 1, wherein said patient has a kidney transplant and/or has a BK virus infection.
Clause 6. The method of clause 1, wherein said patient has a traumatic injury and/or rhabdomyolysis.
Clause 7. The method of clause 1, wherein said patient has exposure to at least one nephrotoxic medicament or toxin.
Clause 8 The method of clause 7, wherein the patient has contrast-induced nephropathy and/or has exposure to a contrast agent and has a high nephropathy risk score.
Clause 9. The method of clause 1, wherein said patient has an infection of the kidney, such as a coronavirus infection, such as SARS-CoV-2, or a polyoma virus infection, such as a BK virus or a JC virus infection.
Clause 10. The method of any one of clauses 1-9, wherein the pharmaceutical composition further comprises a lipid excipient, wherein the pharmaceutical composition optionally is in the form of a lipid nanoparticle.
Clause 11. The method of any one of clauses 1-10, wherein at least one miRNA is an miRNA of the human miRNA-17˜92 cluster, or a mimic of an miRNA of the human miRNA-17˜92 cluster.
Clause 12. The method of any one of clauses 1-11, wherein the at least one miRNA is an miRNA selected from the group consisting of miR-17-3p (SEQ ID NO. 1), miR-17-5p (SEQ ID NO. 2), miR-18a (SEQ ID NO. 3), miR-19a (SEQ ID NO. 4), miR-19b (SEQ ID NO. 5), miR-20a (SEQ ID NO. 6), miR-92a (SEQ ID NO. 7), or a mimic thereof, or any combination thereof.
Clause 13. The method of any one of clauses 1-11, wherein the miRNA, or mimic thereof, is an miRNA-18a mimic, an miRNA-19b mimic, or a combination thereof.
Clause 14. The method of any one of clauses 1-13, wherein the pharmaceutical composition comprises at least two miRNAs, or mimics thereof.
Clause 15. The method of any one of clauses 1-14, wherein the pharmaceutical composition is administered to reduce risk of AKI in said patient,
wherein said patient is in a medical setting or has a medical condition with an increased risk of AKI, and
wherein said patient has not yet begun exhibiting symptoms of AKI.
Clause 16. The method of clause 15, wherein the patient has been administered a contrast agent, and optionally the patient has a high risk of developing AKI as determined by the patient's Mehran score.
Clause 17. The method of any one of clauses 1-14, wherein the pharmaceutical composition is administered to said patient after AKI has occurred.
Clause 18. The method of any one of clauses 1-14, wherein the pharmaceutical composition is administered to said patient before AKI has occurred.
Clause 19. The method of any one of clauses 1-18, wherein the pharmaceutical composition is administered intravenously to said patient.
Clause 20. The method of any one of clauses 1-19, wherein the patient is administered from 0.1 mg to 100 mg of the miRNA or miRNA mimic
Clause 20. The method of any one of clauses 1-19, wherein the patient is administered the pharmaceutical composition at least once per day for at least 5 days.
Clause 21. A pharmaceutical composition comprising:
a miR-18a miRNA, or a mimic thereof;
a miR-19b mRNA, or a mimic thereof, and
at least one liquid, non-aqueous, pharmaceutically acceptable excipient.
Clause 22. Use of miRNA of the miRNA-17˜92 cluster, or an miRNA mimic of an miRNA of the miRNA-17˜92 cluster for treatment or prevention of acute kidney injury.

### BRIEF DESCRIPTION OF THE DRAWING(S)

FIG. 1 illustrates a time course analysis for miR-17˜92 miRNAs and pri-miR-17˜92 in renal endothelial cells at day 1, 3, and 7 post renal IRI. Expression of primary and mature miRNAs in the miR-17˜92 cluster was analyzed by RTqPCR using isolated CD31+ endothelial cells from WT C57BL/6J kidneys 1, 3, or 7 days after renal IRI or uninjured (Un) sham operation. Expression of miR-19a and miR-92 were not detected. Data normalized to Rps17 for pri-miR-17˜92 and U6 snRNA for miRNAs, n=3-11, Tukey multiple comparison, *p<0.05, **p<0.01, ***p<0.001, ****p<0.0001.

FIG. 2 illustrates endothelial-specific knockout of miR-17˜92 exacerbates renal IRI in male mice. FIG. 2A. Serum analysis for blood urea nitrogen (BUN), creatinine (Cr), potassium (K), sodium (Na)/K ratio indicates that renal function is unchanged 1 day but decreased in miR-17˜92^{endo-/-} kidneys 7 days post renal IRI, n=4-6 for uninjured (Un), n=6-9 for renal IRI groups. FIG. 2B. H&E-stained kidney tissues demonstrate that renal tubular injury is exacerbated in miR-17˜92^{endo-/-} mutation kidneys 7 days post-renal IRI. Glomeruli (#), dilated tubules (arrowhead), proteinaceous cast (*), n=5-8. Representative images and semiquantitative scoring for renal tubular damage are shown. FIGS. 2C and 2D. miR-17˜92^{endo-/-} kidneys have increased mRNA (Lcn2) and protein levels of NGAL in whole kidneys 7 days after renal IRI, n=4-8. Arrowheads indicate NGAL+(red)/LTL+(green) injured proximal tubules. Student's t-test, *p<0.05, **p<0.01, ***p<0.001 Scale bars: 50 μm

FIG. 3 illustrates endothelial-specific knockout of miR-17˜92 decreases microvascular function in kidneys after renal IRI. FIG. 3A. Arterial spin labeling (ASL) MRI analysis revealed that renal blood flow is reduced in miR-17˜92^{endo-/-} kidneys 3 days post renal IRI (orange). Representative images for pre-injury and day 3 after renal IRI are shown. MRI signals in renal cortex area with black lines were quantified. FIG. 3B. Endomucin-labelled (red) renal microvasculature is decreased in miR-17˜92^{endo-/-} kidneys 7 days post renal IRI while there is no change in uninjured (Un) kidneys. FIG. 3C. The in vivo oxidative stress marker, isofuran/F2-isoprostanes (IsoP) ratio, is increased in miR-17˜92^{endo-/-} kidneys 7 days post renal IRI. FIG. 3D. F4/80-labeled (brown) infiltrating macrophages are increased in miR-17˜92^{endo-/-} kidneys 7 days post renal IRI. FIG. 3E. There is upregulation of the infiltrating macrophage markers in miR-17˜92^{endo-/-} kidneys 7 days post renal-IRI using RT-qPCR. Data normalized to Rn18S, n=3-8. FIG. 3F. miR-17˜92^{endo-/-} kidneys have increased mRNA (Thbsl) levels of Thrombospondin-1 (TSP1) 7 days post renal IRI, n=4-7. TSP1 is co-localized with renal microvasculature 7 days post renal IRI (arrowheads). TSP1 is also expressed in renal tubular epithelium (*). The dotted box areas are magnified in the images on the right. Student's t-test, *p<0.05, **p<0.01, ***p<0.001. Scale bars: 2.5 mm (A), 100 μm (B), 50 μm (E). Endomucin+ and F4/80+ areas were quantified by tiling imaging methods.

FIG. 4 illustrates the pharmacological co-treatment with miR-18a and miR-19b mimics mitigates renal IRI. FIG. 4A. Schematic for renal IRI model with WT FVB/NJ male mice and mimic treatment regimen (see details for Methods). FIG. 4B. Serum analysis for BUN, potassium (K), and Na/K ratio measured at 7 days post renal IRI indicates that deterioration of renal function is mitigated by co-treatment with miR-18a/ miR-19b mimics administered daily starting 24 hours prior to renal IRI for 8 days while there is no change in uninjured (Un) kidneys, n=4-6. FIG. 4C. H&E-stained kidney tissues demonstrate that renal tubular injury is mitigated by co-treatment of miR-18a/miR-19b mimics when evaluated at 7 days post renal IRI. n=4-6. Glomeruli (#), dilated tubules (arrowhead), proteinaceous cast (*), Representative images and semiquantitative score for renal tubular injury score are shown. FIG. 4D. Endomucin-labelled (brown) renal microvasculature is increased in the mimic treated kidneys 7 days post renal IRI. Glomeruli (#), vascular rarefaction (arrowhead), Student's t-test, *p<0.05, **p<0.01 Scale bars: 50 μm.

FIG. 5 illustrates miR-17˜92^{endo-/-} show endothelial-specific knockout (KO) of miR-17˜92 in kidneys. FIG. 5A. Co-localization of Tie2-Cre expression with Endomucin-labeled renal endothelium are confirmed by using an R26 tdTomato reporter mouse model, n=2. FIG. 5B. miR-17˜92 knockout (KO) allele was detected by PCR in DNA isolated from CD31+ renal endothelium from miR-17˜92^{endo-/-} but not in DNA from the control counterpart (Cre-negative miR-17˜92^{flx/flx} ), Scale bars: 50 μm.

FIG. 6 illustrates High and Low Power Field images of miR-17˜92^{endo-/-} or its controls post renal IRI. The area with dotted line in each LPF image are shown as the HPF image below, n=4-6 for uninjured, n=6-9 for renal IRI groups. The HPF images are identical with the images shown in Figure. 2B. Scale bars: 50 μm.

FIG. 7 illustrates Endothelial-specific knockout of miR-17˜92 increases serum phosphorus and decreases serum bicarbonate post-renal IRI. Serum analysis indicates that renal function is further decreased in miR-17˜92^{endo-/-} mice 7 days post-renal IRI while there is no change in uninjured (Un) mice, n=4-6, Student's t-test, **p<0.01, ***p<0.001

FIG. 8 illustrates Endothelial-specific knockout of miR-17˜92 shows a trend to increase NGAL expression post renal-IRI. Western blotting analysis for NGAL and b-actin (loading control) and its densitometry quantification were shown. Lysates from whole kidneys of miR-17˜92^{endo-/-} or the controls 7 days post renal IRI were used, n=3-4. Student's t-test

FIG. 9 illustrates Endothelial-specific knockout of miR-17˜δ92 exacerbates renal IRI in female mice. FIG. 9A. Serum analysis for blood urea nitrogen (BUN), creatinine, potassium (K), sodium (Na)/K ratio indicates that renal function is decreased in miR-17˜92^{endo-/-} kidneys 7 days post renal IRI, n=3-4 for uninjured, n=2-4 for renal IRI groups. FIG. 9B. Hematoxylin and eosin (H&E) stained kidney tissues demonstrate that renal tubular injury is exacerbated in miR-17˜92^{endo-/-} kidneys 7 days post-renal IRI. n=4-5. Representative images and semiquantitative scoring for renal tubular damage are shown. The area with dotted line in each LPF image are shown as the HPF image below. Glomeruli (#), dilated tubules (arrowhead), proteinaceous cast (*), Student's t-test, *p<0.05, **p<0.01, Scale bars: 50 μm.

FIG. 10 illustrates Cortex areas of left kidneys in ASL MRI images are demarcated by referring to each corresponding anatomical MRI image. Panels A to D. Anatomical magnetic resonance imaging (MRI) images for miR-17˜92^{endo-/-} or Cre negative littermate controls, either pre-injury or 3 days post renal IRI. Anatomical MRI images show that no overt morphological changes are observed between pre and post renal IRI. E-H′. Arterial spin labelling (ASL) MRI images with (6E′-6H′) or without (6E-6H) demarcation of cortex areas of left kidneys. L: Left, Scale bars: 5 mm.

FIG. 11 illustrates that laser-doppler analysis reveals that endothelial-specific knockout of miR-17˜92 shows a trend to decrease of renal blood flow post-renal IRI. Renal blood flow was analyzed with laser-doppler 1 day post renal IRI, n=4-6, Student's t-test

FIG. 12 illustrates angiogenesis associated pathways were predicted to be targeted by miR-17˜92 in renal endothelial cells after renal ischemia/reperfusion injury (IRI). Using the TRAP data for up-regulated genes in renal endothelial cells 24-h post renal IRI, originally published by Liu et al., miR-17˜92 target prediction followed by pathway analysis were performed. (FIG. 12A) Predicted target pathways by miR-17/20, (FIG. 12B) miR-19a/19b, and (FIG. 12C) miR-18a were shown. ERK5 signaling (red colored) was a common pathway amongst all three groups.

FIG. 13 illustrates Renal IRI model with delayed contralateral nephrectomy induces AKI in ischemia time dependent manner at day 7 post injury. Male mice of FVB WT were challenged with different time of renal ischemia (0, 16, 18, or 21 min) FIG. 13A. Serum analysis for blood urea nitrogen (BUN), creatinine, phosphorus, potassium (K), sodium (Na)/ K ratio indicates that renal function is decreased in the kidneys 7 days post renal IRI, n=3-4. FIG. 13B. Hematoxylin and eosin (H&E) stained kidney tissues demonstrate renal tubular injury in the kidneys of FVB WT male mice 7 days postrenal IRI. n=3-4. Representative images and semiquantitative scoring for renal tubular injury are shown. Glomeruli (#), dilated tubules (arrowhead), proteinaceous cast (*), Tukey multiple comparison, *p<0.05, **p<0.01, ***p<0.001, Scale bars: 50 μm.

FIG. 14 illustrates that intravenously administered RNA oligos delivered to the kidneys. FITC-labeled RNA oligos administered were accumulated in renal tubules (arrowheads) (right) but not in PBS-administered kidneys (left), Scale bars: 10 μm.

FIG. 15 illustrates High and Low Power Field images of miR-18a/-19b treated or control-treated kidneys with or without renal IRI at day 7. The area with dotted line in each LPF image are shown as the HPF image below. HPF images are identical with the images shown in FIG. 4. n=4-6, Scale bars: 50 μm.

FIG. 16 is a table with the nucleotide sequences of the miRNAs in the miR-17˜92 cluster.

FIG. 17 is a listing of primer sequences for genotyping miR-17˜92^{endo-/-} mice.

FIG. 18 lists the primer sequences for mRNA RT-qPCR analysis.

FIG. 19 lists the criteria for renal tubular injury scoring.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values.

The articles “a,” “an,” “the,” and “said” are intended to mean that there are one or more of the elements. The terms “comprising,” including,” and “having” are intended to be inclusive and mean that there may be additional elements other than the listed elements.

The term “acute kidney injury” (AKI) refers to an abrupt decrease in kidney function, usually within hours, which encompasses injury to the kidney (structural damage), impairment of the kidney (loss of function), or both. Any clinically-relevant and/or acceptable method and/or marker(s) may be utilized to diagnose AKI. It can be diagnosed by a decrease in the glomerular filtration rate (GFR) as reflected in an acute rise in the serum creatinine levels and/or a decline in overall urine output in a patient. Also, several biomarkers are under investigation for their usefulness in diagnosing AKI, and may be established as useful in identifying AKI in a patient.

“Incipient AM” refers to renal injury as manifested by new-onset proteinuria, cellular activity on urine microscopy, or elevated novel biomarkers of kidney injury in the absence of clinical data that meet current diagnostic criteria for AKI (see, e.g., Perazella MA, et al. Three feasible strategies to minimize kidney injury in ‘incipient AKI’. Nat Rev Nephrol. 2013 August;9(8):484-90). As such, treatment methods described herein can be used to treat incipient AKI. Incipient AKI may include exposure of a patient to contrast agents during angiographic procedures, especially where the patient has a heightened risk of AKI, such as having an elevated Mehran contrast nephropathy risk score (See, e.g., R. Mehran, E.D. et al. A simple risk score for prediction of contrast-induced nephropathy after percutaneous coronary intervention: development and initial validation J Am Coll Cardiol, 44 (2004), pp. 1393-1399 and Mohammed NM, et al. Contrast-induced Nephropathy. Heart Views. 2013;14(3):106-116), or its equivalent. Likewise, patients exhibiting high risk of AKI post-surgery may be evaluated according to other predictors or risk scores, such as in Trongtrakul, K., et al. (Acute kidney injury risk prediction score for critically-ill surgical patients. BMC Anesthesiol 20, 140 (2020)), thereby defining a population having elevated risk of AKI. In some aspects, a patient has a Mehran score greater than 5. In some aspects, the patient has a Mehran score greater than 10. In some aspects, the patient has a Mehran score greater than 15.

The term “miRNA” as used herein is used according to its ordinary and plain meaning and refers to a micro RNA molecule, which may be involved in RNA-based gene regulation (See, generally, O'Brien J, et al. Overview of MicroRNA Biogenesis, Mechanisms of Actions, and Circulation. Front Endocrinol (Lausanne). 2018 Aug. 3;9:402, describing canonical and non-canonical biogenesis of miRNA and miRNA-mediated gene silencing and other mechanisms of action; Macfarlane LA, et al. MicroRNA: Biogenesis, Function and Role in Cancer. Curr Genomics. 2010;11(7):537-561; Liang Chen, et al. Trends in the development of miRNA bioinformatics tools, Briefings in Bioinformatics, Volume 20, Issue 5, September 2019, Pages 1836-1852; Baumann V, et al. miRNA-based therapies: strategies and delivery platforms for oligonucleotide and non-oligonucleotide agents. Future Med Chem. 2014;6(17):1967-1984; and Lam J K, et al. siRNA Versus miRNA as Therapeutics for Gene Silencing. Mol Ther Nucleic Acids. 2015 Sep. 15;4(9):e252). miRNA refers to small single-stranded non-coding fragments of RNA that typically contain 20-25 nucleotides and are found in plants, animals, and some viruses. They can function in RNA silencing and post-transcription regulation of gene expression, and have been implicated in numerous disease states. For example, deletion of the entire miRNA-17˜92 cluster causes skeletal and growth defects in an individual, while other miRNAs have been implicated in, inter alia, cancer, hearing loss, and hereditary keratonconus. Many miRNAs are evolutionarily conserved which suggests important biological functions. Identification of the potential target for a miRNA is often initially done in silico using a variety of different algorithms. This information is then used as a starting point for additional research. Different notational schemes have appeared in the published literature regarding micro RNAs. For example, miRNA-17-92, miRNA-17˜92, miR-17-92, miR-17˜92, miR 17/92, and miR-17-92 have all been used in different sources yet they all refer to the same micro RNA cluster. These different notational formats are considered equivalent herein. Reference to a specific miRNA herein is understood to include naturally-occurring miRNA sequences, such as pre-miRNAs and other miRNA precursors, and isomiRs, that have similar efficacy for the stated use, and includes intra-species allelic variants, mutants, and inter-species variants of the stated sequences.

The term “miRNA mimic” as used herein, refers to a non-naturally occurring molecule similar to miRNA except, for example, the nucleotide sequence of the mimic may be different from a naturally-occurring miRNA, one or more of the natural RNA bases is replaced by a non-natural base, and/or the mimic is an artificial double stranded miRNA fragment (see, e.g., U.S. Pat. Nos. 9,611,478 B2 and 10,584,336 B2, and European Patent No. 2,670,850 B1, incorporated herein by reference for their technical disclosure, for examples of modifications that may be found in miRNA mimics, as well as describing certain miRNAs and miRNA mimics of the miR-17˜92 cluster). The naturally occurring nucleotide bases in RNA are adenine, guanine, cytosine, guanine, and uracil, so any base that occurs in the miRNA that is not one of these four is a non-natural base. Examples of non-natural bases included, but are not limited to, xanthine, purine, 2,6-diaminopurine, 7-deazaadenine, 7-deazaguanine, N⁴N⁴-ethanocytosine, N⁶N⁶-ethano-2,6-diaminopurine, 5-methylcytosine, 5-(C₃-C₆)alkynyl-uracil, 5-(C₃-C₆)alkynylcytosine, 5-fluorouracil or pseudoisocytosine, 2-hydroxy-5-methyl-4-triazolopyrimidine, methylated purines and pyrimidines, and dehydroxylated purines and pyrimidines. Alternatively, a miRNA mimic may comprise an alternate backbone, such as a phosphorothioate, methylated (e.g., 2′-O-methylated), or locked nucleic acid backbones, for example comprising one or more non-ribose sugars. Ribose analogs are often more resistant to hydrolysis and, thus, more resistant to degradation via nuclease activity. Examples of ribose analogs included, but are not limited to, dehydroxylated sugars and rings that omit the ring oxygen atom. Other examples of backbone modifications found in miRNA mimics include substitution of one or more of the phosphate backbone linkages with, for example, an amino acid. In some aspects, the 5′-end of the sequence can have a partially complementary motif (a portion of a longer nucleic acid sequence that has a function) selected to target a sequence within a 3′-UTR (untranslated region) of the target gene. A mimic may be a double-stranded RNA comprising an active strand and a passenger strand, having blunt ends or overhangs. A mimic may or may not structurally resemble a naturally occurring molecule or moiety, but it possesses similar functions to the molecule to which it mimics (See, e.g., Bajan, et al., “RNA-Based Therapeutics: From Antisense Oligonucleotides to miRNAs,” Cells, 9, 137 (2020), doi:10.3390/cells9010137; Zhang SG, et al., “Examination of Artificial MiRNA Mimics with Centered-Site Complementarity for Gene Targeting,” PLoS ONE 8(8): e72062 (2013); and Baumann V, et al., Future Med Chem. 2014;6(17):1967-1984).

The term “therapeutically effective amount” as used herein means a dosage which is sufficient to be effective for the treatment of the patient compared with no treatment. As is known in the art, a therapeutically effective dose of an active agent can vary from patient to patient based on many different factors, including, but not limited to, age, weight, gender, genotype, other medical conditions, etc. A doctor or medical provider overseeing the treatment of a patient is best suited to determine the therapeutically effective dose based on their knowledge and experience working with that patient. A therapeutically-effective amount may be an amount of a therapeutic agent effective to improve one or more symptoms of the disease, or normalize one or more markers of a disease in a patient. By normalize, it is meant to bring values of a marker in a patient towards or into a range considered as normal for a patient.

The term “treatment” or “treat” or “treating” with respect to a disease or medical condition as used herein means the management and care of a patient having developed a disease, condition or disorder. The purpose of treatment is to combat the disease, condition or disorder. Treatment includes, but is not limited to, the administration of a pharmaceutical composition to alleviate one or more symptoms associated with the disease, medical condition or disorder. Treatment may result in the partial or full alleviation of all symptoms, or curing of said disease, medical condition or disorder. With respect to AKI as described herein, treatment can mean that a pharmaceutical composition as described herein is administered to a patient in need of such treatment. A therapeutically effective amount may result in an improvement in a patient's glomerular filtration rate (GFR) or an improvement in biomarkers associated with AKI and may be reflected in a normalization of serum creatinine levels and/or of overall urine output in a patient.

The term “preventing” with respect to a disease or medical condition means that a pharmaceutical composition is administered to a patient before a disease or medical condition has occurred. Typically, with respect to AKI, this means that the patient has a medical condition and/or is in a medical setting (e.g., an intensive care unit at a hospital or being administered a medicament with a known risk of AKI) in which the patient has an increased risk of AKI. Preventing, with respect to AKI, may or may not completely stop AKI from occurring. It may reduce the severity of the AKI as compared to when no pharmaceutical composition is administered to said patient, rather than completely stopping its occurrence.

As used herein, the term “pharmaceutical composition” describes a composition that comprises one or more active agents and one or more pharmaceutically acceptable excipients. The excipient(s) must be acceptable in the sense of being compatible with the other ingredients of the composition and not deleterious to the recipient thereof. The pharmaceutical compositions can be for use in the treatment of any of the conditions described herein, including AKI. In some aspects, the excipient(s) are for use in a parenteral formulation and administration of the active agent(s). In some aspects, the excipient(s) are for use in an intravenous formulation and administration of the active agent(s). In some aspects, the excipient(s) are for use in a subcutaneous formulation and administration of the active agent(s).

Pharmaceutical compositions adapted for parental administration include aqueous and non-aqueous sterile injection solutions which may contain antioxidants, buffers, bacteriostats, and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injection or sterile lipid in oil solution, immediately prior to use. In some aspects the pharmaceutical composition comprises non-aqueous liquid excipients that may optionally be combined with water to form an emulsion.

In one aspect, disclosed herein is a pharmaceutical composition that comprises at least one miRNA, or a mimic thereof, and at least one pharmaceutically acceptable excipient. The pharmaceutical composition may comprise at least two miRNAs, or mimics thereof, wherein each miRNA, or mimic thereof, is selected independently of the other. For example, the composition may comprise one miRNA and one miRNA mimic, or it may comprise two miRNAs, or it may comprise two miRNA mimics.

In some aspects, the miRNA, or mimic thereof, is in the miRNA-17˜92 cluster. The miRNA cluster is well-described in the literature (see, e.g., Mogilyansky, E., et al., “The miR-17/92 cluster: a comprehensive update on its genomics, genetics, functions and increasingly important and numerous roles in health and disease.” Cell Death Differ 20, 1603-1614 (2013)). In some aspects, the miRNA, or mimic thereof, is selected from the group consisting of miR-17-3p (e.g., SEQ ID NO: 1), miR-17-5p (e.g., SEQ ID NO: 2), miR-18a (e.g., SEQ ID NO: 3), miR-19a (e.g., SEQ ID NO: 4), miR-19b (e.g., SEQ ID NO: 5), miR-20a (e.g., SEQ ID NO: 6), miR-92a (e.g., SEQ ID NO: 7), mimics thereof, and combinations thereof. In one nonlimiting example, the composition comprises miR-18a (e.g., SEQ ID NO: 3) and miR-19b (e.g., SEQ ID NO: 5). In another non-limiting example, the composition comprises a miR-18a mimic and miR-19b mimic.

The patient may receive from 0.1 mg to 100 mg of the miRNA, or mimic thereof per dose, including any increment therebetween. When two or more miRNAs, or mimics thereof, are administered, this may be the combined mass of the two or more miRNAs, or mimics thereof, or the mass of each of the two or more miRNAs. The patient may receive, per dose, 0.1 mg, 1 mg, 10 mg, 20 mg, 30 mg, 40 mg, 50 mg, 60 mg, 70 mg, 80 mg, 90 mg, or 100 mg of the miRNA, or mimic thereof. The patient may receive, per dose, approximately or about 0.1 mg, 1 mg, 10 mg, 20 mg, 30 mg, 40 mg, 50 mg, 60 mg, 70 mg, 80 mg, 90 mg, or 100 mg of the miRNA, or mimic thereof.

The patient may receive, per dose, from 0.001 to 1.00 mg/kg of the miRNA, or mimic thereof (based on patient body weight) including any increment therebetween. The patient may receive, per dose, 0.01 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg , or 1.0 mg/kg of the miRNA, or mimic thereof. The patient may receive, per dose, approximately or about 0.01 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg , or 1.0 mg/kg of the miRNA, or mimic thereof.

The pharmaceutical composition comprises a carrier or vehicle that includes at least one pharmaceutically acceptable excipient that is compatible for administration to a human patient. “Excipient” can refer to a pharmaceutically-acceptable material or composition, such as a liquid or solid filler, diluent, manufacturing aid (e.g., lubricant, talc magnesium, calcium or zinc stearate, or steric acid), or solvent encapsulating material, involved in carrying or transporting a therapeutic agent to a patient. Each excipient can be “acceptable” in the sense of being compatible with the other ingredients of the formulation and not injurious to the subject being treated. Some non-limiting examples of materials which can serve as pharmaceutically-acceptable carriers or excipients include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents; (23) serum component, such as serum albumin, HDL and LDL; (24) rheology modifiers; and (25) other non-toxic compatible substances employed in pharmaceutical formulations.

An miRNA and/or miRNA mimic may be delivered using any effective carrier. In one example, the carrier is aqueous, such as water, saline, or PBS. The carrier may be a lipid-based vehicle (See, e.g., Baumann V, et al., Future Med Chem. 2014; 6(17):1967-1984), for example, where at least one excipient may be a non-aqueous liquid. Examples of vehicles employing non-aqueous lipid excipients include, but are not limited to, the MaxSuppressor™ In Vivo RNA-LANCEr II (Lucerna-Chem AG, Luzern, Switzerland) that comprises a neutral lipid, a non-ionic detergent, an oil, and a proprietary mixture of small molecules. The lipid based carrier may be mixed with water to form an emulsion. The specific excipients can be selected based on the mode of administration of the composition and compatibility with the one or more miRNAs, or mimic thereof, present therein. The composition may comprise a lipid based carrier. The lipid based carrier may be suitable for intravenous or subcutaneous administration to the patient. The lipid-based carrier may be a lipid nanoparticle (LNP) composition and/or compositions, e.g., as described in U.S. Pat. Nos. 10,844,028, 10,189,802, 9,872,911, 9,556,110, 9,439,968, 9,227,917, 8,969,353, and 8,450,298, as well as in U.S. Patent Application Publication Nos. 2017/0204075, 2019/0177289, 2017/0152213, 2016/0114042, 2015/0203439, 2014/0322309, 2014/0161830, 2011/0293703, and 2010/0331234, each of which incorporated herein by reference for its technical disclosure relating to compounds and compositions useful in delivery of nucleic acid cargoes, and to the extent it is consistent with the present disclosure. Additional examples of LNPs are described in U.S. Pat. Nos. 9,404,127, 9,364,435, and U.S. Pat. No. 8,058,069, each of which incorporated herein by reference for its technical disclosure relating to compounds and compositions useful in delivery of nucleic acid cargoes, and to the extent it is consistent with the present disclosure (see, also, e.g., Sabnis S, et al., A Novel Amino Lipid Series for mRNA Delivery: Improved Endosomal Escape and Sustained Pharmacology and Safety in Non-human Primates. Mol Ther. 2018; 26(6):1509-1519 and Yonezawa S, et al., Recent advances in siRNA delivery mediated by lipid-based nanoparticles. Adv Drug Deliv Rev. 2020; 154-155:64-78). Examples of lipid nanoparticles and methods of making lipid nanoparticles are described in Whitehead KA, et al., Degradable lipid nanoparticles with predictable in vivo siRNA delivery activity. Nat Commun. 2014 Jun. 27; 5:4277.

Also disclosed herein is a method of treating a human patient, the method comprising administering to the patient a pharmaceutical composition comprising at least one miRNA, or a mimic thereof; wherein said patient has at least one medical condition selected from the group consisting of acute kidney injury (AKI), a medical condition associated with an increased risk of developing AKI, a risk score indicating risk of developing AKI and/or one or more biological markers of AKI, one or more symptoms of AKI, or a sequelae of AKI. Examples of medical conditions associated with AKI include, but are not limited to: renal ischemia, myocardial infarction or heart failure, sepsis, shock, trauma, rhabdomyolysis, kidney transplant, exposure to nephrotoxic medications or toxins, infections including with viruses, and any combination thereof. Hospital-acquired AKI may be caused by surgical procedures, pharmaceutical agents, or diagnostic procedures, especially with the use contrast agents. Patients undergoing procedures or pharmacological treatments associated with high risk of kidney injury, especially patients having one or more comorbidities or risk scores in which there is a high risk of AKI, may be treated as described herein. Kidney transplant patients, and especially patients with BK virus infections may be treated as described herein.

Also disclosed herein is a method of stimulating angiogenesis in a patient. Angiogenesis may be stimulated in a patient's kidneys. The method comprises administering a pharmaceutical composition as described elsewhere herein to a patient in need thereof.

The patient may have AKI. The patient may have an increased risk of developing AKI. An increased risk of AKI is based one or more factors recognized to be associated with AKI. For example, a patient may be receiving a medicament where AKI is a potential complication. A patient may receive a contrast agent that may result in contrast induced-AKI (e.g., contrast-induced nephropathy). A patient may have organ failure syndrome where the kidneys play a role in the development of multi-organ dysfunction. A patient may have received a physical injury that leads to decreased renal perfusion, inflammation and/or blood gas disturbances. A patient may be in an intensive care unit of a hospital and receiving treatment known by medical professionals to have an increased risk of AKI.

In some aspects, the patient may have one or more symptoms or biomarkers suggestive of AKI. Symptoms of AKI include, but are not limited to, decreased urine output, increased creatinine level, decreased GFR, fluid retention, fatigue, irregular heartbeat, chest pain or pressure, seizures, and/or nausea.

The patient may have renal ischemia-reperfusion injury. The patient may have a myocardial infarction, sepsis, or a kidney transplant. The patient may have a traumatic injury or rhabdomyolysis. The patient may have AKI of unknown cause or the cause has not yet been determined. The patient may have a viral infection, such as a coronavirus infection, e.g. a SARS-CoV-2 viral infection (COVID-19), or a polyoma virus infection, such as BK virus or JC virus infection. The patient may have been exposed to at least one nephrotoxic medicament or toxin.

The patient may have sequelae of AKI. Sequelae includes pathological conditions resulting from a disease, injury, therapy, or other trauma. For AKI, there are three common pathological sequelae (excluding, or other than, full recovery, in which case no treatment is necessary): 1) incomplete recovery of renal function resulting in chronic kidney disease, 2) exacerbation of pre-existing chronic kidney disease progressing toward renal failure, and 3) end stage renal failure. A patient having received an AKI, regardless of etiology, may not completely recover from an AKI and may have at least one pathological sequelae associated therewith.

The timing of administering the pharmaceutical composition to the patient may be based on the medical needs of the patient as determined by the health care professional overseeing the treatment of said patient. The patient may have already exhibited one or more symptoms or markers associated with AKI so the composition is administered in order to treat the existing AKI. The patient may not exhibit one or more symptoms or markers associated with AKI but presents a medical condition or is in a medical setting associated with an increased risk of developing an AKI. In that case, the patient is treated with the pharmaceutical composition in order to prevent AKI or to reduce the severity of AKI.

The patient may be administered a pharmaceutical composition as described elsewhere herein. A patient may be administered one or more miRNAs, miRNA mimics, or combination thereof. At least one miRNA may be an miRNA of the miRNA-17˜92 cluster, or mimic of an miRNA of the miRNA-17˜92 cluster. At least one miRNA may be chosen from miR-17-3p, miR-17-5p, miR-18a, miR-19a, miR-19b, miR-20a, miR-92a, mimics thereof, or combinations thereof.

The miRNA, or mimic thereof, is a miRNA-18a mimic, a miRNA-19b mimic, or a combination thereof.

### EXAMPLES

### Methods

### Mouse Model

C57BL/6J wild-type (WT), Tie2 Cre (FVB-Tg(Tek-cre)^{2352Rwng/J}, R26-tdTomato Cre reporter (B6.Cg-Gt(ROSA)26Sor^{tm14(CAG-tdTomato)Hze}/J, and miR-17˜92 floxed (Mirc1^{tm1.1Tyj}/J) mice were used in these experiments. Tie2 Cre; R26-tdTomato reporter mice, which harbor heterozygous Tie2 Cre and R26 tdTomato reporter alleles, were generated. Endothelial-specific miR-17˜92 knockout, which harbors heterozygous Tie2 Cre and homozygous floxed alleles of miR-1 7˜92 (miR-17˜92^{endo-/-} ), were also generated. The offspring were genotyped by PCR with the primer sequences in FIG. 17. The primer pair F4/R5 (to amplify 255-bp miR-17˜92 wide type allele or the 289-bp miR-17˜92 floxed allele fragment), and the primer pair F4/AV145 (to amplify the 441-bp miR-17˜92 deleted allele fragment) were used. Tie2 Cre negative-, sex-, and age-matched littermates of miR-17˜92^{endo-/-} mice were used as controls. The genetic background of miR-17˜92^{endo-/-} and the control mice are C57BL/6J. For the miRNA mimics experiments, WT FVB/NJ males were used. Male mice were used unless otherwise noted.

Isolation of Endothelial Cells from Mouse Kidneys

CD31+ endothelial cells from mouse kidneys were isolated from a single cell suspension of kidney cells obtained from 10-14 week-old, male, miR-17˜92^{endo-/-} or Cre-negative littermate control mice using the Dynabeads Antibody Coupling Kit conjugated to a CD31 antibody.

### IRI Models in Mice

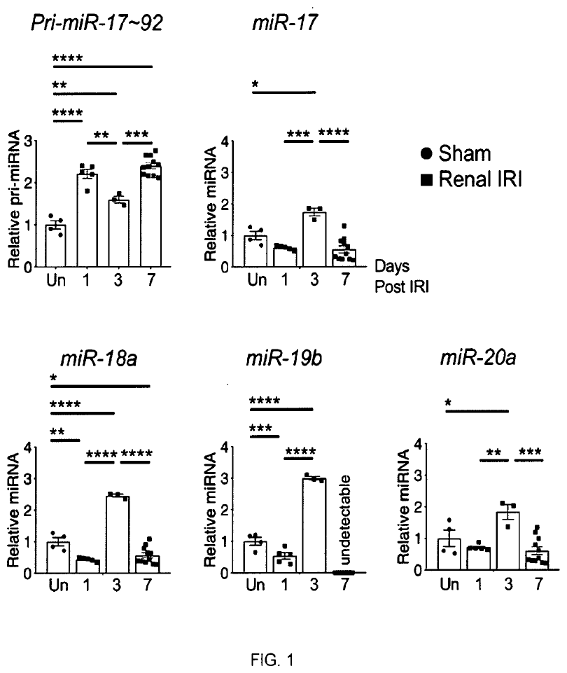

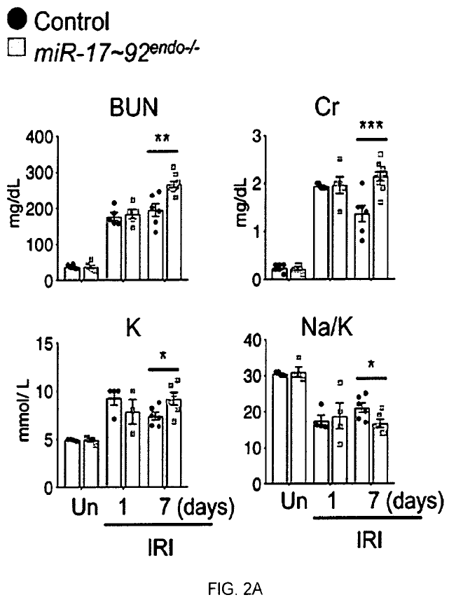

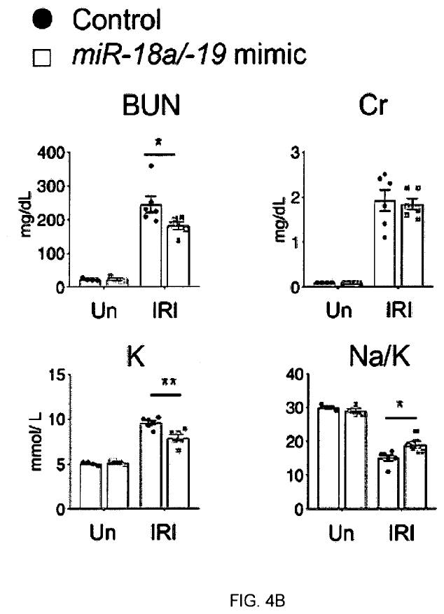

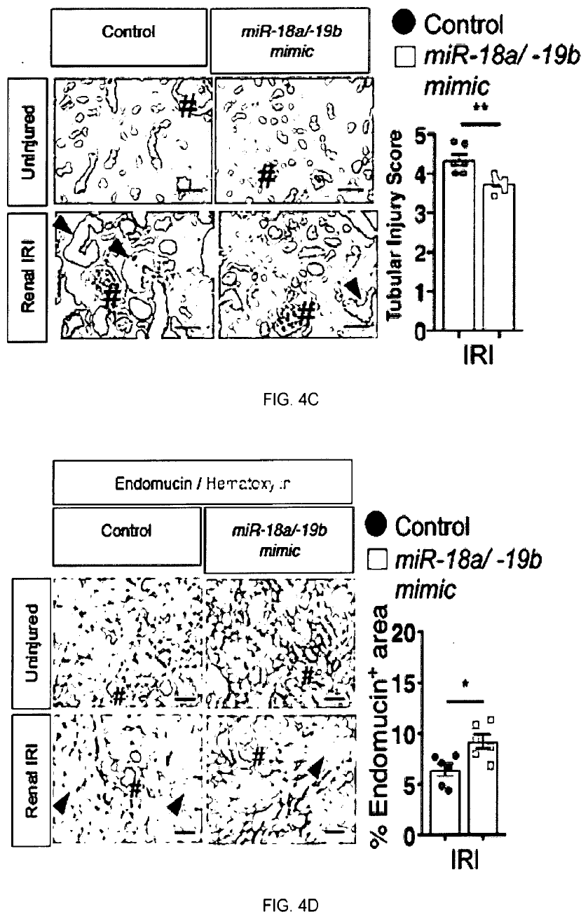

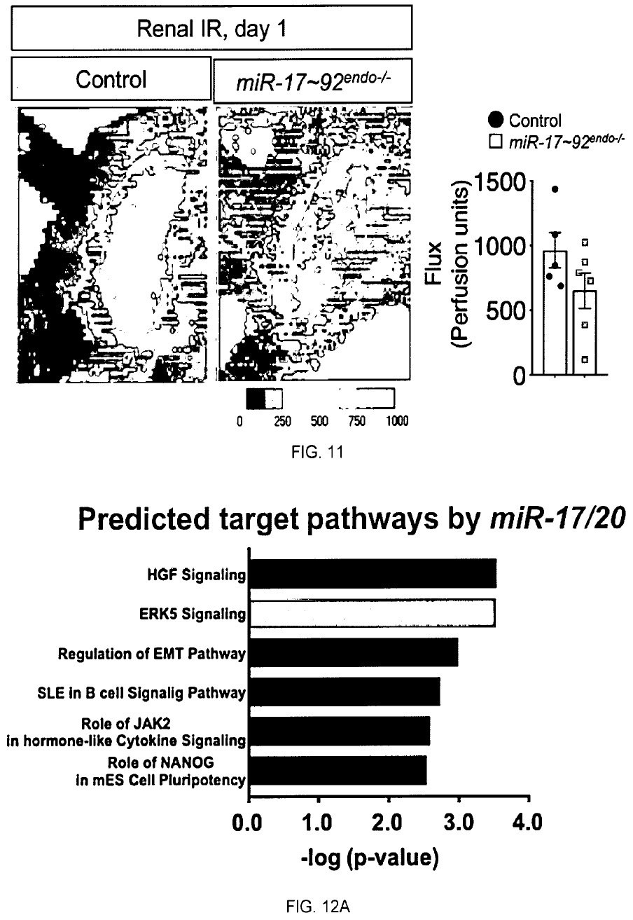

To induce ischemic AKI, a renal IRI model was performed as described in FIG. 4A. Mice were anesthetized with the inhalant 2% isoflurane. Core body temperature of the mice was monitored with a rectal thermometer probe and was maintained at 36.8-37.2° C. throughout the procedure with a water heating circulation pump system and a heat lamp. Buprenorphine was administered for pain control (0.1 mg/kg body weight, subcutaneous). With aseptic technique, a dorsal incision was made to expose the kidney. Renal ischemia was induced by unilateral clamping of the left kidney pedicle with a non-traumatic microvascular clamp. Length of ischemia was optimized as 22 min for WT C57BL/6J males for expression analysis of miR-17˜92 miRNAs (FIG. 1), 22 min for males and 31 min for females of miR-17-92^{endo-/-} or Cre-negative littermate control, and 21 min for WT FVB/NJ males for the miRNA mimic experiments. The 21 min ischemia time for WT FVB/NJ males was determined following a series of ischemia titration experiments (FIG. 11). Reperfusion was visually verified. Delayed contralateral nephrectomy of the right kidney was performed 1 day prior to harvesting samples. Mice were sacrificed on day 7 to harvest blood and the remaining kidney Sham procedure followed the same protocol except for the step of renal pedicle clamping. Serum was separated from the blood and analyzed for the levels of creatinine, blood urea nitrogen (BUN), potassium, sodium, phosphorus, and bicarbonate.

miRNA Mimics Treatment

All miRNA mimics were purchased from Dharmacon and formulated with Max-Suppressor in vivo RNALancerll, a lipid-based delivery reagent. The sequences are for the mimics were:

hsa-miR-18a:
(SEQ ID NO: 3)
uaaggugcaucuagugcagauag;
and
hsa-miR-19b:
(SEQ ID NO: 28)
ugugcaaauccaugcaaaacuga.

Combined miRNA mimics miR-18a (C-310513-07-0050) / miR-19b (C-311179-00-0050) or control mimics (0.25 mg/kg bw iv) were administered via tail vein. The C. elegans miRNA, cel-miR-67, based miRNA mimic (CN-001000-01-50) was used for control. The miRNA mimic or control were administered daily starting 24 hours prior to renal IRI for 8 days (FIG. 4A). This vehicle, dose and daily treatment regimen were effective in a mouse cardiac infarction model.

For the biodistribution analysis, a 100 μL of 10% FITC labeled RNA oligo (BLOCK-iT™ Fluorescent Oligo, Thermo Fisher, 13750062) or PBS control was administered via tail vein injection. Kidneys were harvested 40 min post-injection and processed for cryosections.

Magnetic resonance imaging (MRI) analysis

Cortex areas of the left kidneys in mice were longitudinally analyzed by in vivo MRI non-invasively for anatomical and functional evaluation before and 72 hours post renal IRI. T₂-weighted MRI images were used for anatomical images. Rate of renal blood flow (RBF) was quantified by arterial spin labeling (ASL) MRI. ASL uses the endogenous water molecules in the blood as tracers to quantify RBF without needing to administer exogenous contrast agents. A continuous adiabatic pulse train in a 1 mm-slab localized 9 mm to the renal artery was applied to the abdominal aorta to spin-tag all the incoming proton spins through the abdominal aorta to reach the steady state. The spin attenuation by the steady-state labeling, labeling efficiency, blood-tissue partition coefficient, and T_{1obs} were measured to calculate the RBF with the following equation:

RBF (mL/100g/min)=λ*(T_{1obs}*2α)⁻¹*(M_{C}−M_{L})⁻¹
λ: blood-tissue partition coefficient=0.9 mL/l
T_{1obs}: Observed or apparent T₁ value (ms)
M_{C}: control magnetization
M_{L}: labeling magnetization
α: labeling efficiency
α was measured in the abdominal aorta 0.9 cm from the arterial spin inversion site.

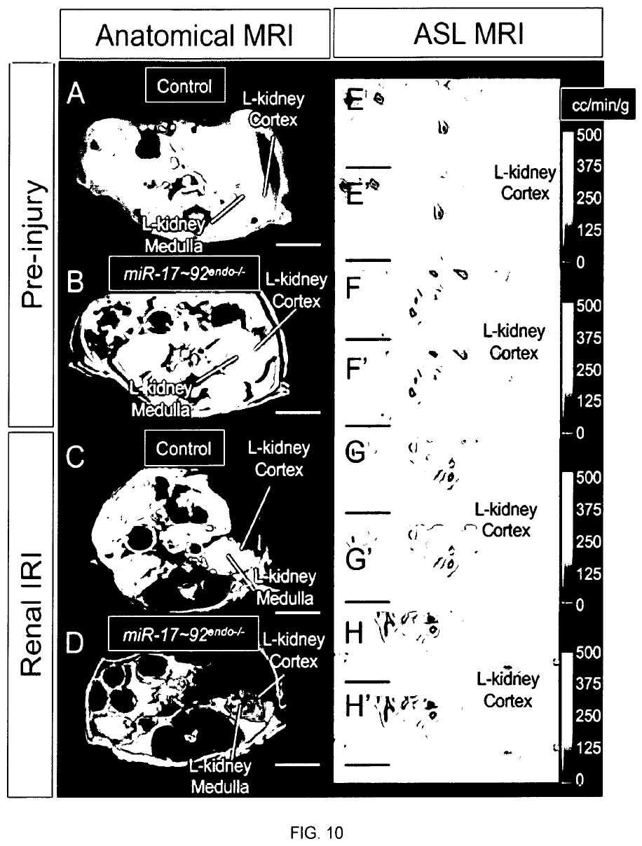

Anatomical MRI images were referred to define cortex area of the kidneys in ASL MRI images (FIG. 10).

### Laser-Doppler Blood Flow Analysis

Renal blood flow was assessed 24 h after renal IRI. Mice were anesthetized with 2.5% isoflurane and placed in a supine position on a heating pad. Core temperatures were maintained at 36° C. and continuously monitored by rectal probe thermometer. Left kidneys were exposed with abdominal incisions and real-time blood flow was measured using laser Doppler imaging.

miRNA Target Prediction and Target Pathway Analysis

The translating ribosome affinity purification (TRAP)-microarray dataset for up-regulated genes in renal endothelial cells 24-h post renal IRI was obtained from NCBI Gene Expression Omnibus (GEO), GSE2004, originally published by Liu, et al., TargetScan (www.targetscan.org/vert_72/) webserver was used for miRNA target prediction. Ingenuity Pathway Analysis (IPA, Qiagen) was used for pathway analysis.

Real-time quantitative PCR analysis (RT-qPCR)

Kidneys were homogenized and total RNA, including miRNA, was isolated using the miRNeasy Mini or Micro Kit. For miRNA analysis, cDNA was reverse-transcribed from 10 ng of total RNA with TaqMan MicroRNA Reverse Transcription Kit. For pri-miR-17-92 analysis, cDNA was reverse-transcribed from 20 ng of total RNA with High-capacity cDNA reverse transcription kit. RT-qPCR analysis was performed with TaqMan® Small RNA Assay probes, TaqMan® Universal PCR Master Mix No AmpErase® UNG, and CFX96 Touch™ Real-Time PCR Detection System with C1000 Thermal Cycler. Cycling conditions were 95° C. for 10 minutes, then 40 cycles of 95° C. for 15 seconds and 60° C. for 1 minute. As endogenous controls, U6 snRNA was used to normalize expression of miR-17, miR-18a, miR-19a, miR-19b-1, miR-20a, and miR-92a-1 while Rps17 was used for pri-miR-17-92. Expression was analyzed using the 2^{-ΔΔCt} method.

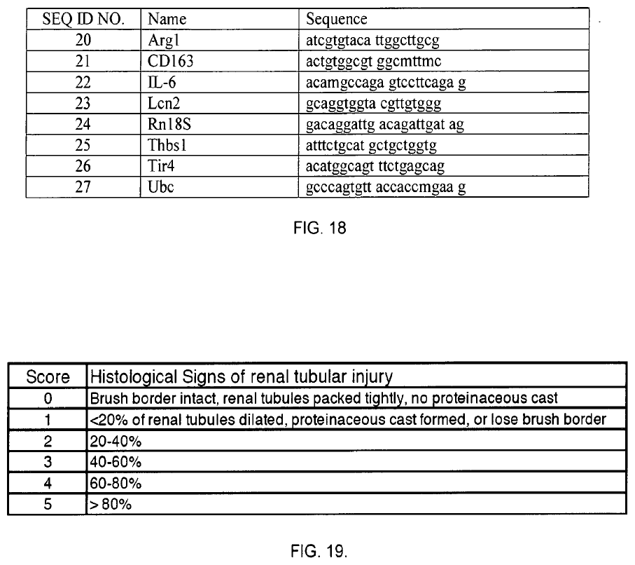

For mRNA analysis, cDNA was reverse-transcribed from 500 ng of total RNA with SuperScript First-Strand Synthesis System II with primers of Oligo dT and random hexamers. RT-qPCR analysis was performed with gene specific primer oligos (FIG. 18), SsoAdvanced™ SYBR® Green Supermix, and CFX96 Touch™ Real-Time PCR Detection System with C1000 Thermal Cycler. Cycling conditions were 95° C. for 10 minutes, then 40 cycles of 95° C. for 15 seconds and 60° C. for 1 minute. Rn18S was used for endogenous control, which expression was normalized to using the 2^{-ΔΔCt} method.

### Tissue Analysis

Kidneys were fixed in 4% paraformaldehyde and embedded in paraffin or OCT. The paraffin embedded tissues were sectioned at 4 microns. Hematoxylin & eosin (H&E) staining was performed for histology evaluation. Semi-quantitative blinded scoring (0-5) for tubular injury was performed in terms of tubular dilation, proteinaceous cast formation, and loss of brush border with microscopic fields at 40× magnification that cover the entire renal cortex with an AX-500 microscope. The criteria are described in FIG. 19. The representative images for the figures were obtained with a Leica DM2500 optical microscope and its DFC 7000T camera.

Immunolabelling was performed. OCT-mounted frozen tissue sections at 10 micron thickness were used for TSP1 immunostaining. For other antigens, antigen retrieval of deparaffinized 4 micron sections was performed with 10.0 mM sodium citrate (pH=6) boiled with microwave for 15 min. Tissues were labeled with 1:200 rat anti-Endomucin (V.7C7) (Santa Cruz, sc-65495), 1:50 rat anti-Neutrophil gelatinase-associated lipocalin (NGAL) (Abcam, ab70287), 1:50 mouse monoclonal anti-TSP1(A6.1) biotin (MA5-13395), and 1:1000 rat anti-F4/80 (NB600-404) with 1:200 secondary antibodies of donkey anti-Rat IgG Alexa Fluor 594 (A-21209), donkey anti-Rat IgG Alexa Fluor 488 (A-21208) or NeutrAvidin Dylight 594 (22842). 1:100 FITC-conjugated Lotus Tetragonolobus Lectin (LTL) (FL-1321) were used to co-label with NGAL. F4/80-labeled tissues were visualized with the Bond Polymer Refine Detection System (DS9800). The representative images for the figures were obtained with a Leica DM2500 optical microscope and its DFC 7000T camera.

To quantify Endomucin+, F4/80+, or NGAL+ area in a tissue section, systems for tiling imaging were used to capture the entire field of an immunolabelled kidney section with 20× objectives Immunostaining-labeled area in corticomedullary region was quantified with NIS Elements software. To quantify microvasculature in kidneys, Endomucin⁺-glomeruli were manually excluded from the images. All tissue evaluation was performed in a blinded fashion.

### Western Blotting

The mouse kidneys were collected and immediately snap-frozen. The protein homogenates derived from the whole kidney tissues were immunoblotted with 1:200 goat anti-NGAL antibody. The immunoblot images for the figures was obtained with a ChemiDoc XRS+ System.

### Isoprostane and Isofuran Measurement

F2-isoprostanes (IsoP) and isofurans were measured after lipid extraction from snap-frozen kidney samples using gas chromatography-mass spectrometry.

### Statistical Analysis

Data are presented as mean±SEM. Prism 7.0C software was used for statistical analysis. To determine whether sample data has been drawn from a normally distributed population, D'Agostino-Pearson Omnibus Test or Shapiro-Wilk test was performed. For parametric data, Student's t-test was used to compare two different groups. Tukey multiple comparison test was used to compare multiple groups. For non-parametric data, Mann-Whitney U test was used. The threshold of p<0.05 was set to consider data statistically significant.

### Results

The levels of all 6 miRNAs in isolated CD31⁺ renal endothelial cells from WTC57BL/6J mouse kidneys after renal IRI or sham operation (FIG. 1) was analyzed. miR-17, miR-18a, miR-19b and miR-20a expression were down-regulated or unchanged at day 1, up-regulated and peaked at day 3, and down-regulated at day 7 post-renal IRI. Pri-miR-17-92 expression was up-regulated throughout the time course when compared to the sham controls. The expression pattern of pri-miR-17-92 was inversely correlated to miR-17, -18, -19b, and -20a miRNAs, consistent with post-transcriptional regulation of the production of these mature miRNAs.

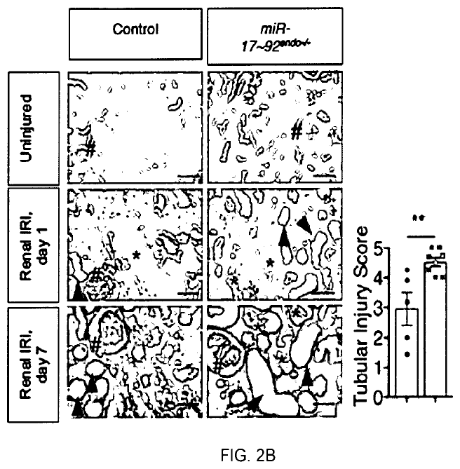

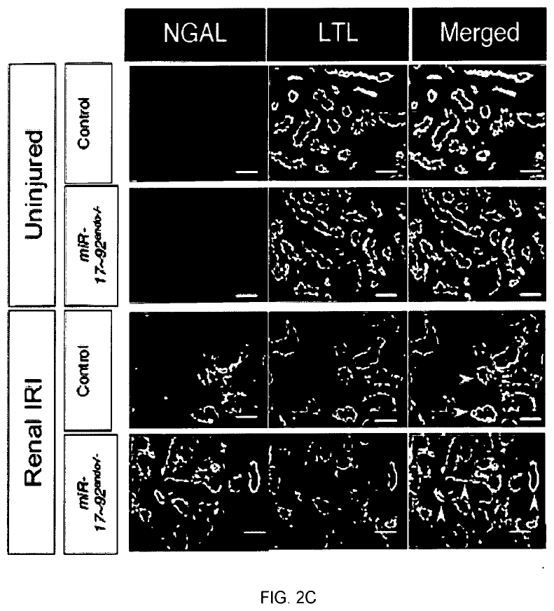

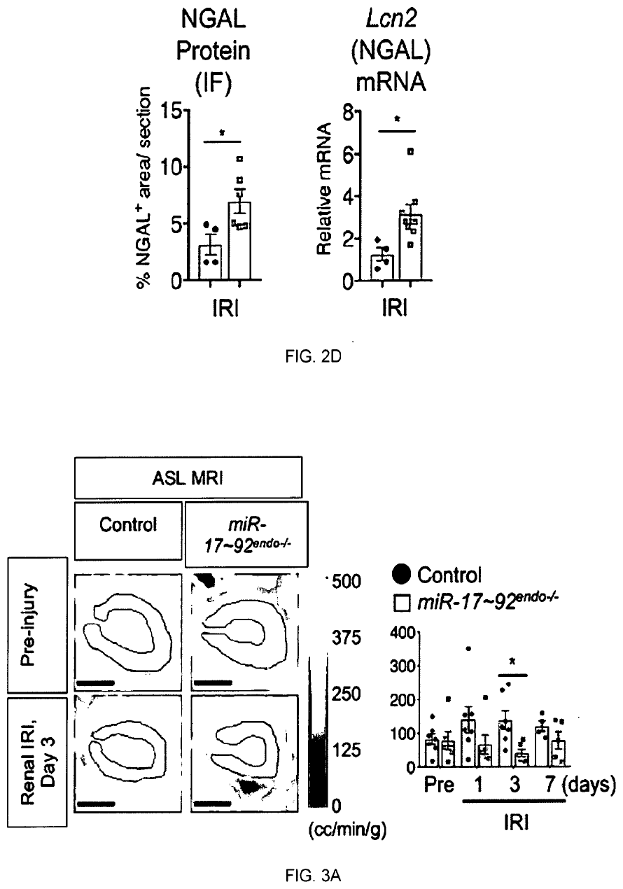

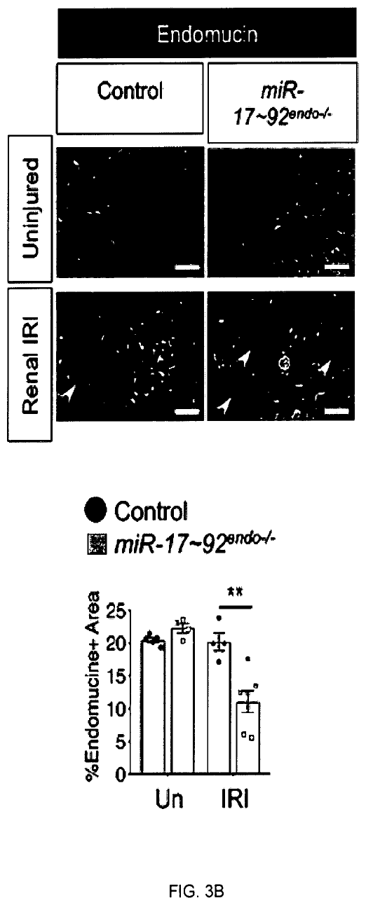

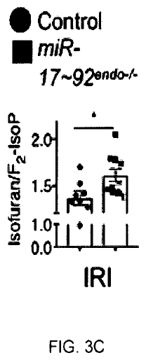

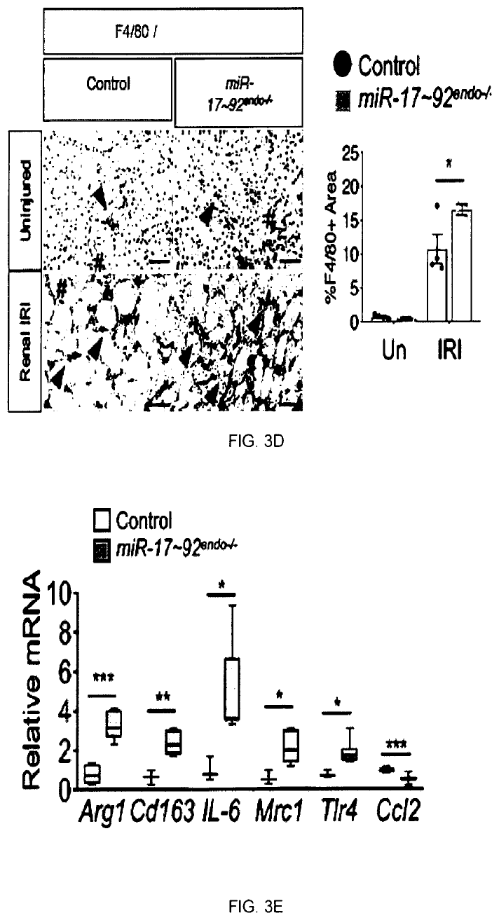

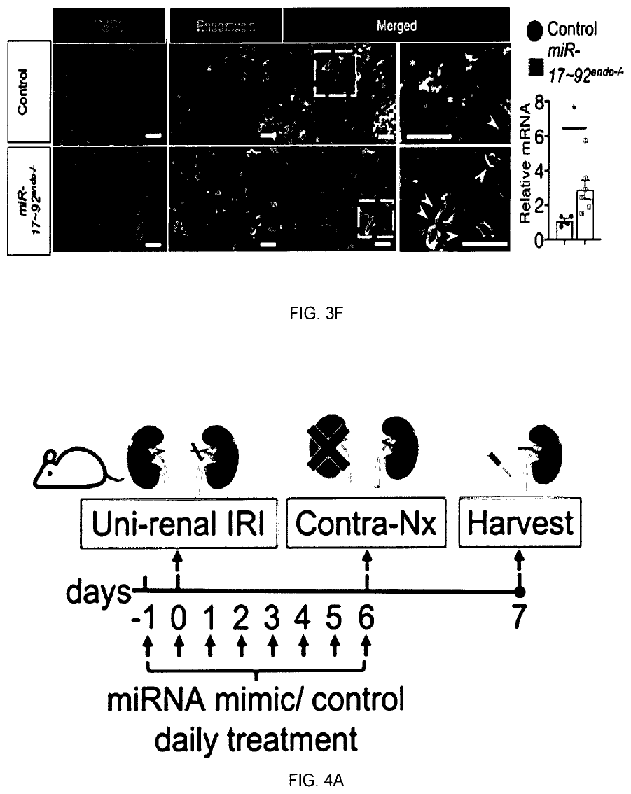

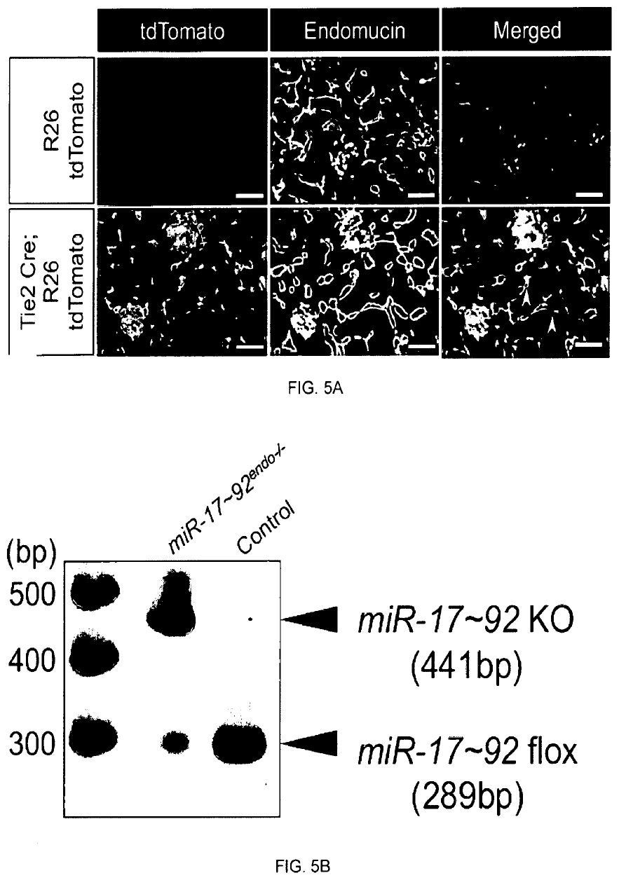

Because miR-17˜92 is a pro-angiogenic factor, a mouse model of Tie2 Cre-mediated, endothelial-specific miR-17˜92 knockout (hereafter referred to as: miR-17-92^{endo-/-} ) was generated. It was confirmed that Tie2 Cre was expressed in renal endothelium by co-localized expression of its R26-tdTomato reporter and an endothelial marker, endomucin, and that miR-17-92endo−/− mediated efficient knockout of miR-17˜92 in renal endothelium (FIG. 5). No difference was noted in renal function, renal vasculature, or the amount of F4/80⁺ resident macrophages of uninjured miR-17-92^{endo-/-} mice compared to controls (FIG. 2A-B, FIG. 3A, B, D).

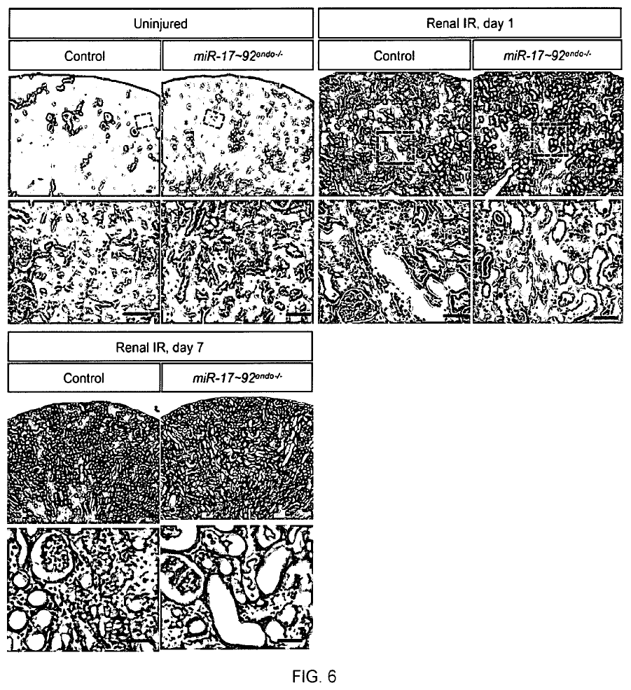

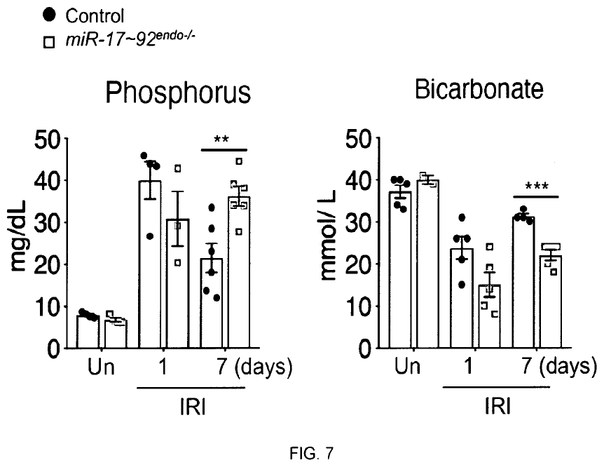

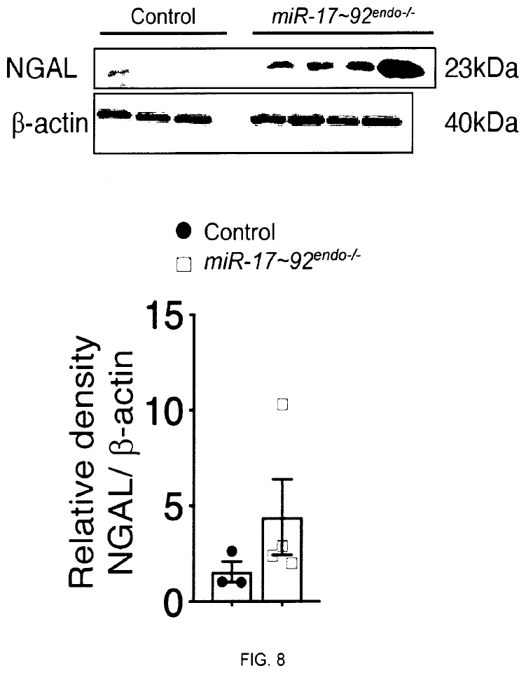

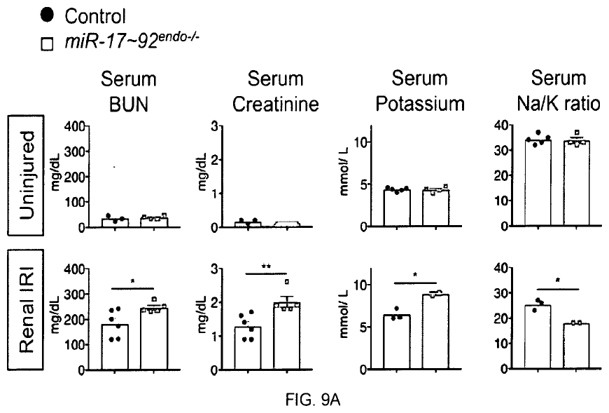

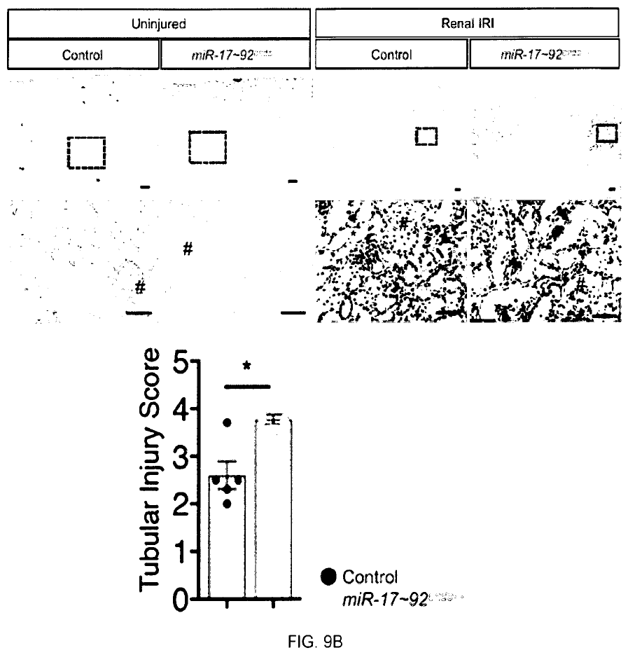

Renal IRI on male miR-17-92^{endo-/-} and Cre-negative littermate controls was then performed. miR-17-92^{endo-/-} had increased renal injury 7 days post-renal IRI, assessed by increased serum levels of BUN, creatinine and potassium, with a decrease in Na/K ratio (FIG. 2A), as well as increased serum phosphorus and decreased serum bicarbonate (FIG. 7). H&E-stained renal tissue confirmed the presence of increased renal tubular injury in miR-17-92^{endo-/-} kidneys at day 7 (FIG. 2B, arrowheads, FIG. 6). NGAL is an injury marker for kidney tubules in mice and humans. It is expressed in a punctate cytoplasmic pattern in both proximal tubules and in multiple cell types in the distal segments. Levels of NGAL mRNA (Lcn2) and protein were increased in miR-17-92^{endo-/-} kidneys 7 days post-renal IRI (FIGS. 2C, 2D, FIG. 8). There were more widespread NGAL⁺ LTL⁺ proximal tubular epithelial cells in miR-17-92^{endo-/-} compared to controls (FIG. 2C, arrowheads). Consistent results were obtained with the female mice (FIG. 9). These changes were not observed at 1 day post-renal IRI, suggesting that endothelial-miR-17˜92 act in the later phase of renal IRI.

Ischemic AKI in mice is also characterized by reduction of renal blood flow and microvascular rarefaction, which is often associated with incomplete endothelial repair after injury. It was thought that miR-17-92^{endo-/-} impairs the function of the renal vasculature. ASL MRI, which allows non-invasive/longitudinal evaluation of renal blood flow with its excellent quantitative capacity, demonstrated that the cortex areas of miR-17-92^{endo-/-} kidneys had reduced renal blood flow 3 days post renal-IRI (FIG. 3A). A consistent trend towards a decrease in renal blood flow at day 1 post renal IRI by ASL MRI was confirmed by laser-doppler analysis (FIG. 11). Consistent with this, endomucin-labeled renal microvasculature was decreased in miR-17-92^{endo-/-} 7 days post-renal IRI, when compared to controls (FIG. 3B).

Endothelial damage by renal IRI increases infiltration of inflammatory leukocytes including macrophages to the kidneys and promotes AKI. F4/80⁺macrophages are increased in in miR-17-92^{endo-/-} kidneys (FIG. 3D). Furthermore, RT-qPCR analysis revealed that multiple macrophage markers of M1 and M2 types were increased in miR-17-92^{endo-/-} kidneys 7 days post-renal IRI (FIG. 3E). mRNA levels of a monocyte recruiting factor, Ccl2, was down-regulated in miR-17-92^{endo-/-} kidneys. These data may suggest that infiltrated monocytes are stuck in the kidney and are unable to exit from the miR-17-92^{endo-/-} kidneys at day 7 due to enhanced microvascular damage. Oxidative stress induces endothelial senescence that limits angiogenesis, and miR-17˜92 is known to limit reactive oxygen species generation in lung cancer cells. We measured the isofuran/F2-IsoP ratio, a well-established in vivo oxidative stress marker. The isofuran/F2-IsoP ratio was increased in miR-17-92^{endo-/-} kidneys 7 days post renal IRI (FIG. 3C), suggesting that lack of endothelial-derived miR-17˜92 increases renal oxidative stress post renal IRI. Together, these data suggest that miR-17-92^{endo-/-} kidneys exacerbate functional impairment of renal vasculature after renal IRI.

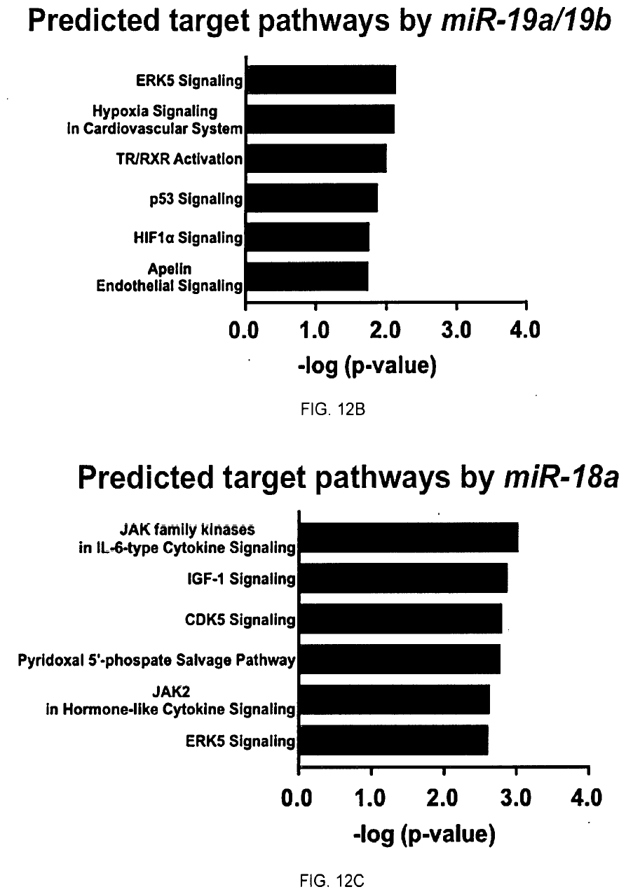

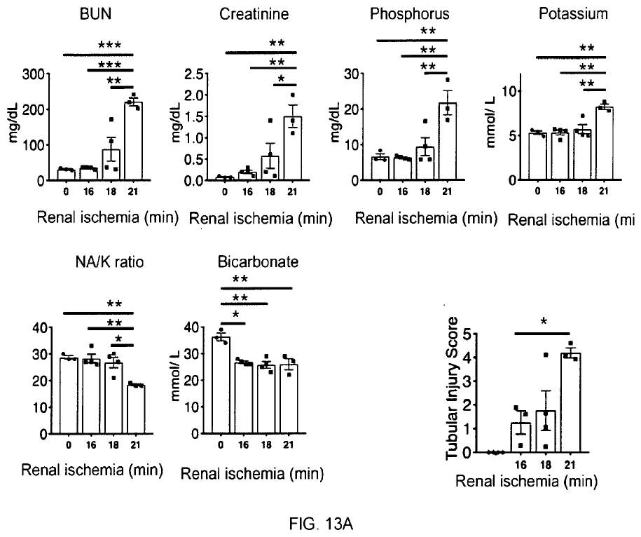

Next, the putative down-stream target genes for miR-17˜92 in the renal endothelium during renal IRI was explored. Using a previously published TRAP-microarray data set of up-regulated genes in renal endothelial cells 24 h post renal IRI, we performed a computational prediction for miR-17˜92 targets using TargetScan 7.1. Focusing on the 4 miR-17˜92 members that were up-regulated after renal IRI, we identified 407 predicted genes that are targeted by miR-17/20 (183), miR-18a (44), and/or miR-19a/b (180). Pathway analysis for the predicted miR-17˜92 targets revealed that multiple angiogenesis-associated pathways, including extracellular signal-regulated kinase 5 (ERK5), p53, and HIF1α signaling, were suggested to be regulated by miR-17˜92 in renal endothelial cells after renal IRI (FIG. 12). ERK5 was a common downstream pathway for targets of miR-17/20, miR-18a, and miR-19a/b, and is known to negatively regulate VEGF signaling Furthermore, TSP1 is a potent anti-angiogenic factor and well characterized miR-17˜92 target. This computational prediction also identified TSP1 as a target for miR-18a and miR-19a/b. Consistent with this, Thbs1 (TSP1) mRNA was increased in miR-17-92^{endo-/-} kidneys 7 days post-renal IRI (FIG. 3F) and TSP1 protein was highly expressed in endomucin-labeled renal microvasculature (FIG. 3F, arrowheads). This suggests that miR-17˜92 suppresses anti-angiogenic factors and increases the angiogenic capacity of renal endothelial cells in response to renal IRI.

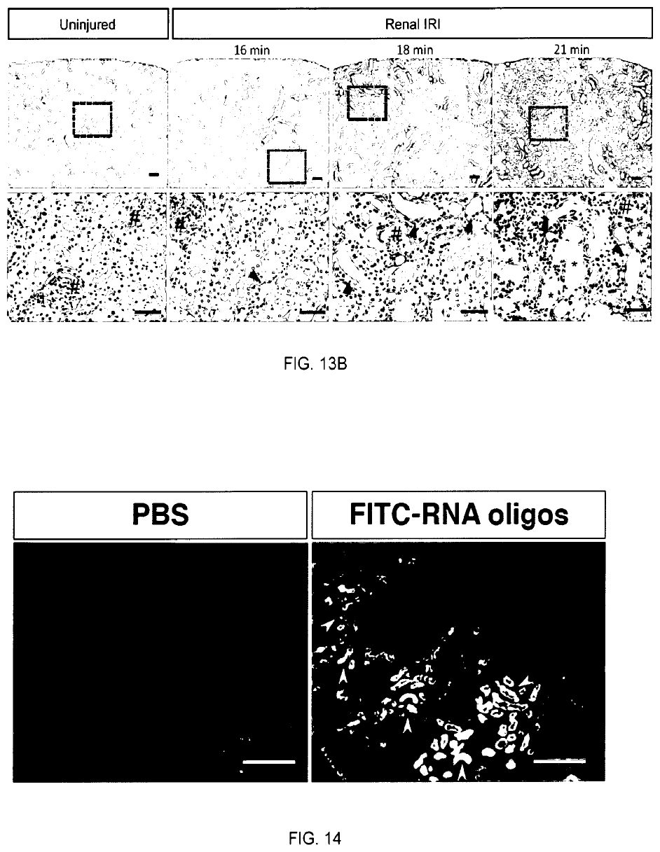

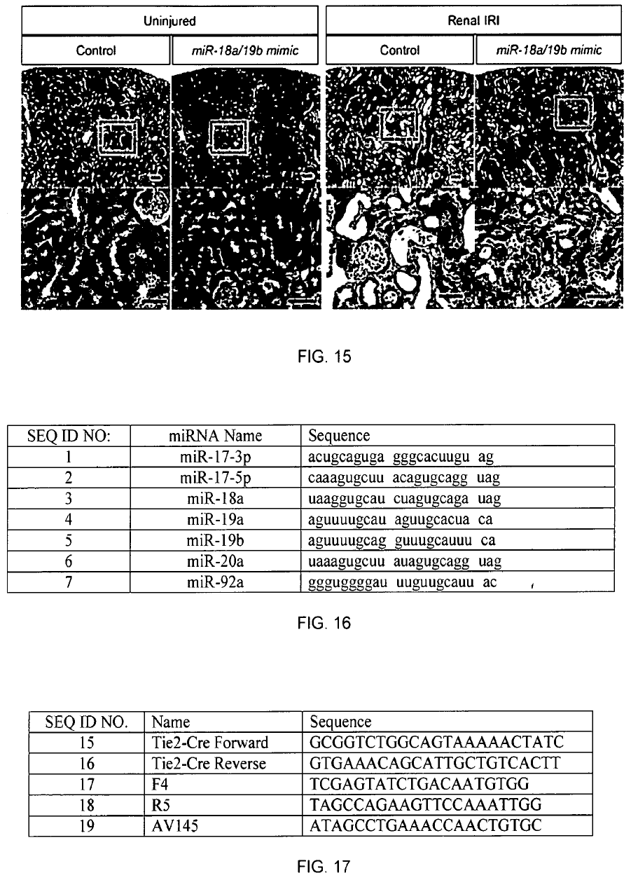

This data illustrates that miR-18a and miR-19b were the most highly expressed miR-17˜92 family members in renal endothelial cells 3 days post renal IRI (FIG. 1). This suggests that pharmacological co-treatment of miRNA mimics for miR-18a and miR-19b mitigates renal IRI. Biodistribution of FITC-labeled RNA oligos to the kidneys was confirmed (FIG. 14). Additionally, daily co-treatment of miR-18a/miR-19b miRNA mimics for WT FVB/NJ male mice, starting 24 hours prior to renal IRI for 8 days, decreases serum levels of BUN and potassium, increases Na/K ratio, and reduces renal tubular injury (FIG. 4A-C). Endomucin-labeled renal microvasculature was increased in miR-18a/-19b treated kidneys, suggesting that the mimics act on the renal microvasculature for its beneficial effects (FIG. 4D).

The present study revealed that lack of endothelial-derived miR-17˜92 results in impaired endothelial function and stimulates expression of a potent anti-angiogenic factor, TSP1, in the renal microvasculature. Consistent with the angiogenic role of miR-17˜92 known only in vitro or cancer settings, our novel in vivo findings demonstrate the angiogenic capacity of miR-17˜92 in the setting of renal IRI.

The examples and embodiments included herein are illustrations only. A person skilled in the art will recognize that alternative equivalent embodiments are also within the scope of the subject matter herein.

1. A method of treating a human patient, the method comprising:
administering to the patient a pharmaceutical composition comprising at least one miRNA of the miRNA-17˜92 cluster, or an miRNA mimic of an miRNA of the miRNA-17-92 cluster;
wherein said patient has at least one medical condition chosen from acute kidney injury (AKI), a medical condition associated with an increased risk of developing AKI, incipient AKI, or a sequelae of AKI.

2. The method of claim 1, wherein said patient has renal ischemia.

3. The method of claim 1, wherein said patient has a myocardial infarction, congestive heart failure, sepsis, or shock.

4. (canceled)

5. The method of claim 1, wherein said patient has a kidney transplant and/or has a BK virus infection.

6. The method of claim 1, wherein said patient has a traumatic injury and/or rhabdomyolysis.

7. The method of claim 1, wherein said patient has exposure to at least one nephrotoxic medicament or toxin, the patient has contrast-induced nephropathy, and/or has exposure to a contrast agent, and optionally has a high nephropathy risk score.

8. (canceled)

9. The method of claim 1, wherein said patient has an infection of the kidney, such as a coronavirus infection, such as SARS-CoV-2, or a polyoma virus infection, such as a BK virus or a JC virus infection.

10. The method of claim 1, wherein the pharmaceutical composition further comprises a lipid excipient, wherein the pharmaceutical composition optionally is in the form of a lipid nanoparticle.

11. The method of claim 1, wherein the at least one miRNA is an miRNA of the human miRNA-17˜92 cluster, or a mimic of an miRNA of the human miRNA-17-92 cluster.

12. The method of claim 1, wherein the at least one miRNA is an miRNA selected from the group consisting of miR-17-3p (SEQ ID NO. 1), miR-17-5p (SEQ ID NO. 2), miR-18a (SEQ ID NO. 3), miR-19a (SEQ ID NO. 4), miR-19b (SEQ ID NO. 5), miR-20a (SEQ ID NO. 6), miR-92a (SEQ ID NO. 7), or a mimic thereof, or any combination thereof.

13. The method of claim 1, wherein the miRNA, or mimic thereof, is an miRNA-18a mimic, an miRNA-19b mimic, or a combination thereof.

14. The method of claim 1, wherein the pharmaceutical composition comprises at least two miRNAs, or mimics thereof.

15. The method of claim 1, wherein the pharmaceutical composition is administered to reduce risk of AKI in said patient,
wherein said patient is in a medical setting or has a medical condition with an increased risk of AKI, and
wherein said patient has not yet begun exhibiting symptoms of AKI.

16. The method of claim 15, wherein the patient has been administered a contrast agent, and optionally the patient has a high risk of developing AKI as determined by the patient's Mehran score.

17. The method of claim 1, wherein the pharmaceutical composition is administered to said patient after AKI has occurred.

18. The method of claim 1, wherein the pharmaceutical composition is administered to said patient before AKI has occurred.

19. The method of claim 1, wherein the pharmaceutical composition is administered intravenously to said patient.

20. The method of claim 1, wherein the patient is administered from 0.1 mg to 100 mg of the miRNA, miRNA mimic.

20. (canceled)

21. A pharmaceutical composition comprising:
a miR-18a miRNA, or a mimic thereof;
a miR-19b mRNA, or a mimic thereof, and
at least one liquid, non-aqueous, pharmaceutically acceptable excipient.

22. (canceled)

23. The method of claim 1, wherein the patient is administered the pharmaceutical composition at least once per day for at least 5 days.
